(19) United States
(12) Patent Application Publication
(10) Pub. No.: US 2026/0004680 A1
(43) Pub. Date: Jan. 1, 2026

(54) POINT-OF-CARE ULTRASOUND (POCUS) CARDIORESPIRATORY ARREST TRAINER (POCUS CAT)

(71) Applicant: TRAINING BRIDGE INC., Hudson, NY (US)

(72) Inventor: Peter Marotta, Hudson, NY (US)

(21) Appl. No.: 19/253,144

(22) Filed: Jun. 27, 2025

Related U.S. Application Data
(60) Provisional application No. 63/665,278, filed on Jun. 28, 2024.
(60) Provisional application No. 63/665,277, filed on Jun. 28, 2024.

(51) Int. Cl. G09B 23/28; G16H 40/63

(52) U.S. Cl. CPC G09B 23/286; G16H 40/63

(57) ABSTRACT

A multi-functional training system for ultrasound, auscultation, and pressure-based clinical procedures is disclosed. The system includes at least one pressure sensing layer and surface or near-surface references to determine the location, orientation, and applied pressure of devices such as ultrasound probes and stethoscopes. A computing unit, executes software modules to receive sensor data, determine positioning, and provide visual and feedback outputs. The system supports realistic simulation across diverse patient profiles and scenarios, including neonatal, pediatric, adult, geriatric, and animal forms. The system facilitates accurate training aligned with clinical protocols such as Advanced Cardiac Life Support (ACLS) and FAST/eFAST, and enables tracking of multiple devices and limb elements simultaneously. An optional second pressure layer allows force vector and absolute pressure measurement for use in palpation, hemorrhage control, and object placement training. The system improves procedural training fidelity through integrated feedback on device positioning, orientation, and force application.

### CROSS-REFERENCES

Pub. No.: U.S. Pat. No. 10,573,201 B2, Pub. Date Feb. 25, 2025
Pub. No.: U.S. Pat. No. 9,386,960 B2, Pub. Date Jul. 12, 2016
Pub. No.: US 2023/0223140 A1 (43) Pub. Date: Jul. 13, 2023
Pub. No.: U.S. Pat. No. 11,813,111 B2, Pub. Date Nov. 14, 2023
Pub. No.: U.S. Pat. No. 11,819,292 B2, Pub. Date Nov. 21, 2023
Pub. No.: US 2024/0008845 A1, Pub. Date: Jan. 11, 2024
Eun S, Yoon H, Kang S Y, Jo I J, Heo S, Chang H, Lee G, Park J E, Kim T, Lee S U, et al. Real-Time Tracheal Ultrasound vs. Capnography for Intubation Confirmation during CPR Wearing a Powered Air-Purifying Respirator in COVID-19 Era. Diagnostics. 2024; 14 (2): 225. https://doi.org/10.3390/diagnostics14020225
Michael Gottlieb, Stephen Alerhand, Managing Cardiac Arrest Using Ultrasound, Annals of Emergency Medicine, Volume 81, Issue 5, 2023, Pages 532-542, ISSN 0196-0644, https://doi.org/10.1016/j.annemergmed.2022.09.016. (https://www.sciencedirect.com/science/article/pii/S0196064422011209)
Michael Gottlieb, MD, Stephen Alerhand, MD, Managing Cardiac Arrest Using Ultrasound, Cardiology/expert clinical management| Volume 81, ISSUE 5, P532-542, May 2023
Wong, A., Vignon, P. & Robba, C. How I use ultrasound in cardiac arrest. Intensive Care Med 49, 1531-1534 (2023). https://doi.org/10.1007/s00134-023-07249-8
Bughrara N, Diaz-Gomez J L, Pustavoitau A. Perioperative Management of Patients with Sepsis and Septic Shock, Part II: Ultrasound Support for Resuscitation. Anesthesiol Clin. 2020 March; 38 (1): 123-134. doi: 10.1016/j.anclin.2019.11.001. PMID: 320086407.
Ávila-Reyes, D., Acevedo-Cardona, A. O., Gómez-González, J. F. et al. Point-of-care ultrasound in cardiorespiratory arrest (POCUS-CA): narrative review article. Ultrasound J 13, 46 (2021). https://doi.org/10.1186/s13089-021-00248-0
Maite A Huis In't Veld, Michael G Allison, David S Bostick, Kiondra R Fisher, Olga G Goloubeva, Michael D Witting, Michael E Winters, Ultrasound use during cardiopulmonary resuscitation is associated with delays in chest compressions, DOI: 10.1016/j.resuscitation.2017.07.021
Hussein L, Rehman M A, Sajid R, Annajjar F, Al-Janabi T. Bedside ultrasound in cardiac standstill: a clinical review. Ultrasound J. 2019 Dec. 30; 11 (1): 35. doi: 10.1186/s13089-019-0150-7. PMID: 31889224; PMCID: PMC6937355.
Huis In't Veld M A, Allison M G, Bostick D S, Fisher K R, Goloubeva O G, Witting M D, Winters M E. Ultrasound use during cardiopulmonary resuscitation is associated with delays in chest compressions. Resuscitation. 2017 October; 119:95-98. doi: 10.1016/j.resuscitation.2017.07.021. Epub 2017 Jul. 25. PMID: 28754527.
Blanco P, Martínez Buendía C. Point-of-care ultrasound in cardiopulmonary resuscitation: a concise review. J Ultrasound. 2017 Jul. 31; 20 (3): 193-198. doi: 10.1007/s40477-017-0256-3. PMID: 28900519; PMCID: PMC5573702.
Gaspari R, Weekes A, Adhikari S et al (2016) Emergency department point-of-care ultrasound in out-of-hospital and in-ED cardiac arrest. Resuscitation 109:33-39
Hwang S O, Zhao P G, Choi H J et al (2009) Compression of the left ventricular outflow tract during cardiopulmonary resuscitation. Acad Emerg Med 16 (10): 928-933
Shin J, Rhee J E, Kim K (2007) Is the inter-nipple line the correct hand position for effective chest compression in adult cardiopulmonary resuscitation? Resuscitation 75 (2): 305-310
Arntfield R, Pace J, Hewak M et al (2016) Focused transesophageal echocardiography by emergency physicians is feasible and clinically influential: observational results from a novel ultrasound program. J Emerg Med 50 (2): 286-294

### BACKGROUND OF THE INVENTION

There is a need in the training environment to use devices like ultrasound on manikin or simulated humans to reduce risks to actual humans during the training on various locations on human anatomy. Current systems limit the range of training on simulated humans by restricting mobility training events and the ability to use either a real ultrasound device or simulated device.

Training on live patients puts patients at risk for negative outcomes which raise both legal and ethical issues for the practitioner. The case representations that a practitioner is seeing is limited to the people they scan and so the exceptions that can put a patient at risk are understudied by practitioners and thus place patients at higher risk.

However, there is no inherent internal anatomy in CPR capable manikins in existence, so there is no way for any practitioner to practice the procedure prior to the invention. The current method of providing internal anatomy in manikins as outlined by BluePhantom patents is a static presentation and does not support CPR chest compressions nor chest compression evaluation, it will also not provide any indication between asystole or very fine ventricle fibrillation. Bluephantom relies on making internal structures that mirror human anatomy but not physiological changes that occur during the function of the human body, under normal or abnormal states.

The procedure outlined to evaluate the status of the heart during CPR has not been widely adopted because there is currently no training equipment that integrates all the key aspects of the procedure into one trainer. Wider adoption of the procedure to be incorporated into existing American Heart Association (AHA) professional level CPR and Advanced Cardiac Life Saving certification standards requires that practitioners demonstrate a functional skill and knowledge to be certified and qualified to conduct the procedure. The cohort of professionals require this certification include all, but not limited to the following, (medical doctors, nurse practitioners, physician assistance, mid wives all levels of nursing, medical specialist like respiratory therapy, all paramedics, EMT's). The AHA requires that mechanical skills for certification be demonstrated on manikins not real patients. The manikin system needs to be able to provide feedback to the student during training and collect data for the instructor to perform post training debriefing and evaluation of the students based on best practices of simulation. Since all US medical facilities and health service providers that have staff performing professional level CPR and/or ACLS require documentation of certification, the need creates a requirement to have a manikin-based training system for wider adoption of the POCUS-CAT procedure in the US and globally. Research indicates that with training, that currently 30% of CPR compressions can be improved to increased survivability of victims and up to 25% of patients that are currently considered in asystole or presumed dead, can be revived from an actual reversible cardiac state but currently are not.

U.S. Pat. Nos. 10,573,201 B2 and 9,386,960 B2 are two patents from a competitive product that actually tries to mimic the internal anatomy as part of their trainers. They go into the actual construction of how they replicate the internal anatomy structures to be scanned and the body in which they are molded into approximate human anatomy. The patents expressly focus on not replicating the internal human anatomy but rather using images and video of real or fabricated scans which are oriented in such a way to align probes' orientation. This is an external process that combines images. There are several key advantages: model lasting longer, greater variability able to scan during the movement of patient like in real world and or during procedures like CPR for are a few.

Ávila Reyes et al. summarizes that, “When POCUS-CA is used, studies have shown that 10 to 35% of patients with asystole have a demonstrable cardiac contraction.”

“In general, the ROSC rate was greater than 50% if the cardiac activity was detected with ultrasound.”

“In a multicenter study published in the “Resuscitation Journal” in the United States in 2016 (Gaspari R et al.) “REASON STUDY” included 793 patients with out-of-hospital cardiac arrest who presented non-defibrillable rhythms, cardiac activity was present in 33% of the patients. It was associated with ROSC″.

“Adequate or high-quality compressions are associated with ROSC and survival. Literature reports improper hand position during resuscitation may lead to compression of the ascending aorta, aortic root, or outflow tract of the left ventricle, but not the left ventricle.” (Hwang S O et al.; Shin J et al.)

“Constant visualization of the heart during compressions allows an objective assessment of resuscitation quality, determining the proper location of the hands of the provider of CPR to avoid obstruction of the left ventricular outflow tract and allowing adequate ventricular relaxation, which guarantees an adequate preload and cardiac output.” (Arntfield R et al.) (Reference-Supporting research for need of the use of ultrasound by trained professionals is discussed research of Avila Reyes et al. The Ultrasound Journal (2021) 13:46, https://doi.org/10.1186/s13089-021-00248-0)

Other use cases include the use of ultrasound on limbs, such areas as head, arms and legs and attachments. In this case a reference with the AC electromagnetic internal references will be made along with internal gyro/accelerometer/time of flight to determine rotation, pitch and yaw of the limb relative to the body and internal reference from a defined device reference in the limb or attachment. This will then be aligned with reference data from the device, ultrasound probe, or other medical devices used with or independently similarly referenced as the ultrasound tracking clip. This will allow but limited to things like chest needle decompression, intravenous catheter insertions, evaluation for deep vein thrombosis, and crushed limb syndrome as examples. Other devices that can configured with the system using an imbedded or clip on style would include but not limited to needle and catheter systems, auscultation devices, intubation devices, pulse oximetry devices, capnography devices, medical and rescue devices used on or in proximity to the manikin patient or simulated patient or provider. These uses and devices can be used with but not limited to CPR, POCUS or other medical or clinical events for training that would be trained to do in real medical, health interventions or practice.

Research has shown how an ultrasound device is used during a suspected cardiac event, or CPR/ACLS procedures can cause longer delays in chest compressions. The training system must train active POCUS scans during compressions and minimize time during which compressions are paused to do select scans and then resume the cardiac protocols and compressions.

Current strategies for training focus on the quality of compressions in a very specific location on the chest with limited evaluation of an electrocardiogram (ECG), capnography and palpation of pulse points. Research has shown that in live cardiac events, Ultrasound allows for a new view of what quality compressions can be as well as determination of fine ventricular fibrillation that an ECG will not detect. Research also suggests that the Ultrasound can provide details on other compounding factors such as fluids that act in a cardiac tamponade, clots and/or pulmonary embolisms, pneumothorax, infections, pulses, blood flow and device location such as airways which are all critical to improving the patient's survival rate during a cardiac event. Ultrasound training equipment currently available on the market does not allow for dynamic assessment during a cardiac event or present altered human anatomy from normal variations like heart placement, body mass of the patient, or trauma of infection.

This training system supports Basic Life Saving (BLS) and Advanced Life Saving (ALS) levels of interventions, as well as those procedures or protocols that rely on these as elements of their training or implementation, such as Advanced Cardiac Life Saving ACLS protocols, extended field care and others.

### SUMMARY OF THE INVENTION

This training system and method supports treatment strategies Point-of-Care Ultrasound in cardiorespiratory arrest (POCUS-CA) that build on American Heart Association (AHA), Advanced Cardiac Life Saving (ACLS) protocols. Clinicians typically use POCUS at patients' bedside for quick evaluations of conditions like heart failure or abdominal pain. Focused Assessment with Sonography for Trauma (FAST) ultrasound exams, meanwhile, are quick imaging procedures that utilize high-frequency sound waves to create images of the body's internal structures. All other clinical use of ultrasound evaluations is frequently referred to as “general ultrasound.” The present invention allows for consistent quality training of POCUS in support of protocols like ACLS and focuses on assessment with ultrasonics for Trauma FAST/eFAST. This system supports neonatal, pediatric youth/young adult, adult and geriatric for female and male forms or representations and varying body types for height, weight, various skin tones and body configurations as well as use in animal forms such as but not limited to canine resuscitations.

Further, this system and method determine the location and orientation of the device to the surface of simulated body or body part and to maintain the reference as the device is moved along the surface. Based on the orientation reference that an image or information is presented to show what a scan or the device would present if used in the same manner as a real human being. This system and methodology have been developed to support both active devices and simulated devices. The system and method allow for the accurate reference and orientation of devices relative to a body or limb to include but limited to the movement of the body and limbs including such things as the movement of the chest wall, the movement of limbs by people training, rolling and positioning the patient. That this tracking and orientation can support single or multiple devices simultaneously. Current tracking allows up to a combined 16 device and limb elements per internal reference, but that number can increase with newer systems or with more than one internal reference.

### FIELD OF THE INVENTION

The present invention relates to a system and method relating to training.

### BRIEF DESCRIPTION OF THE FIGURES

FIG. 1 shows an example of probe movement and orientation for compression and fine fibrillation detection.

FIG. 2 shows the typical Point of Care Ultrasound Scan (POCUS) that requires rapid real-time alignment to provide realistic scan. (Ávila Reyes et al. The Ultrasound Journal (2021) 13:46 Page 4 of 14).

FIG. 3 shows best practices for POCUS. Light rectangles represent areas checked during chest compressions. Dark rectangles represent areas checked during pulse check.

FIG. 4 shows the relationship of the probe to the body surface and an internal structure with reference objects. For a real ultrasound device, the return reflection will determine the angle and distance to each of the reference objects, which can be used to calculate the orientation and position of the probe relative to the surface of the body. Based on that information, an image that has been recorded or simulated can be presented and oriented to what would appear on a scan of a real human. Objects that are positioned or placed in the body during the scan can be presented so that the final image is a compost made in real time to show a procedure that is conducted on the body.

FIG. 5 shows the relationship of a simulated probe in relationship to the body surface and an internal structure with reference objects. Additional references for the probe are surface or near surface references such as RFID tags and external reference and internal to the device during flight or gyroscopic reference. The internal reference structures can also be used as receivers if a beam is created from the simulated device. Used in combination or in part, such a device can calculate the orientation and position of the probe relative to the surface of the body. Based on that information, an image that has been recorded or simulated can be presented and oriented to what would appear on a scan of a real human. Objects that are positioned or placed in the body during the scan can be presented so that the final image is a composite made in real time to show a procedure that is conducted on the body.

FIG. 6 shows the relationship of a simulated probe in relationship to the body surface and an internal structure with reference objects. Additional references for the probe are surface or near surface references such as RFID tags and external reference and internal to the device during flight or gyroscopic reference. The internal reference structures can also be used as receivers if a beam is created from the simulated device. Used in combination or in part, such a device can calculate the orientation and position of the probe relative to the surface of the body. Based on that information, an image that has been recorded or simulated can be presented and oriented to what would appear on a scan of a real human. Objects that are positioned or placed in the body during the scan can be presented so that the final image is a composite made in real time to show a procedure that is conducted on the body.

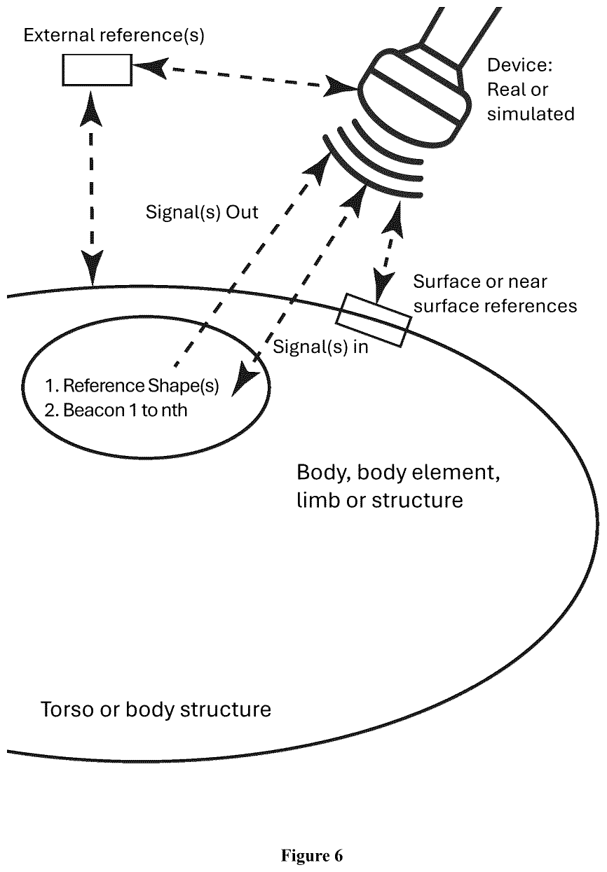

FIG. 7 shows an outline of the relationships of the surfaces and operational structures along with optional elements that may be used for the different operational modes outlined below with the pressure sensing layer providing a relationship of the general operational parts and optional parts that may be used to enhance the simulation for a given clinical simulation.

FIG. 8 shows the relationship of the device with the clip and probe in relationship to the body with the AC electromagnetic internal reference

FIG. 9 shows the simulation system with simulated ultrasound probe.

FIG. 10 shows the simulation system using a real ultrasound probe.

FIG. 11 shows the relationship of the device with the clip and probe in relationship to the body with the AC electromagnetic internal reference.

FIG. 12 shows a simulation system using a real or simulated ultrasound probe.

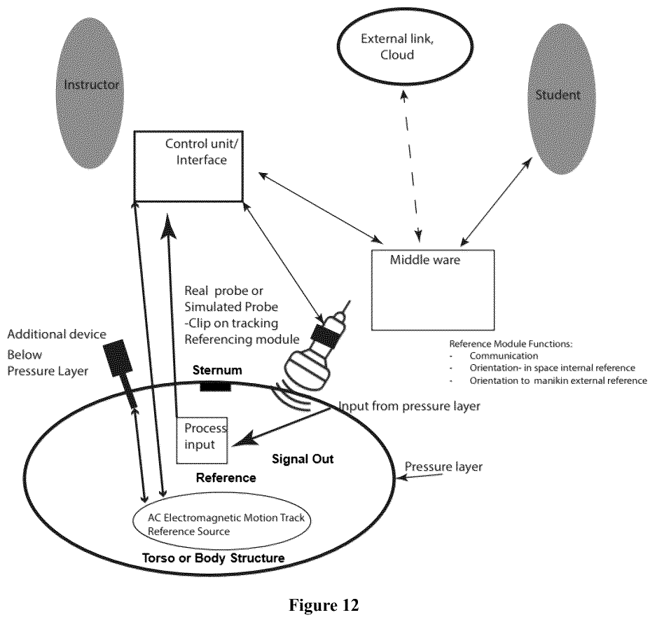

FIG. 13 simulation system using a real or simulated ultrasound probe.

FIG. 14 shows the configuration of a tracking clip that would fit over a real or simulated probe.

FIG. 15 shows an active probe clip mounted next to a theoretical real or simulated probe.

FIG. 16 shows the grid orientation in which pressure is applied to the pressure sensitive layer.

FIG. 17 shows the determination of force vectors using the pressure sensing layer.

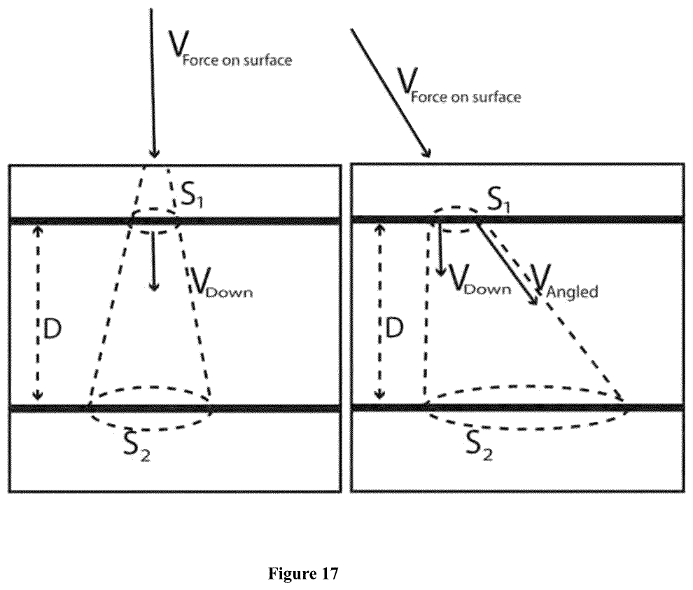

### DETAILED DESCRIPTION OF THE INVENTION

This invention provides a system and method with reduced harm and risks to patients relative to live training on humans directly. Current training using multiple ultrasound guided needle insertions to learn how to do it safely increases the risk of infection, improper device placement, and failed procedures. For practitioners it reduces confirmation bias against patients since it offers a wider range of training outside of the normal band that a practitioner may routinely see.

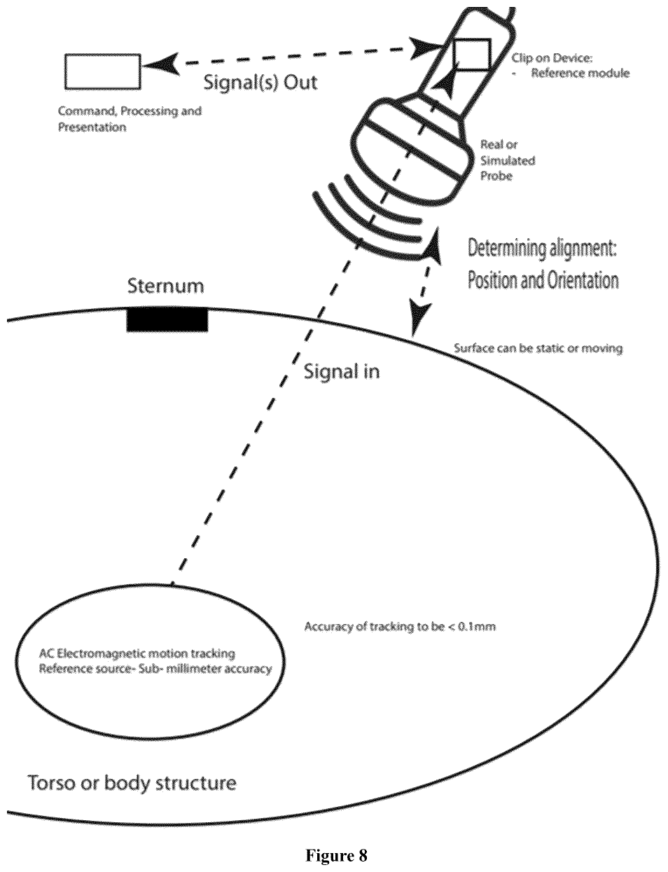

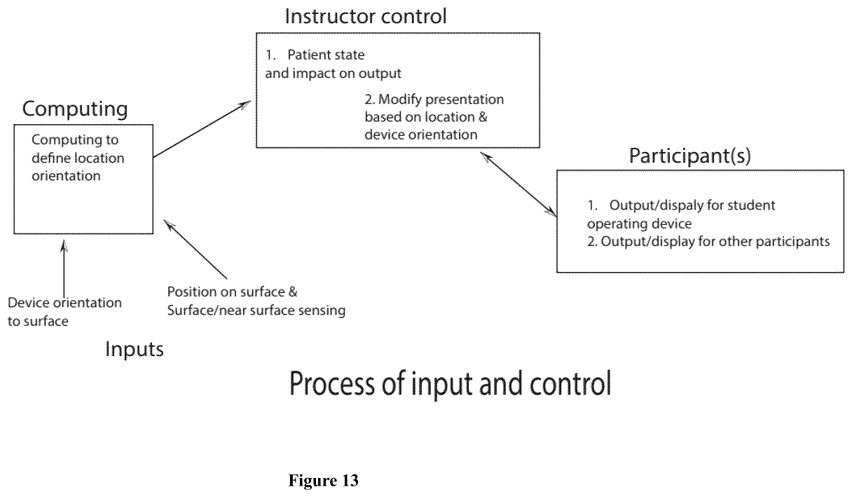

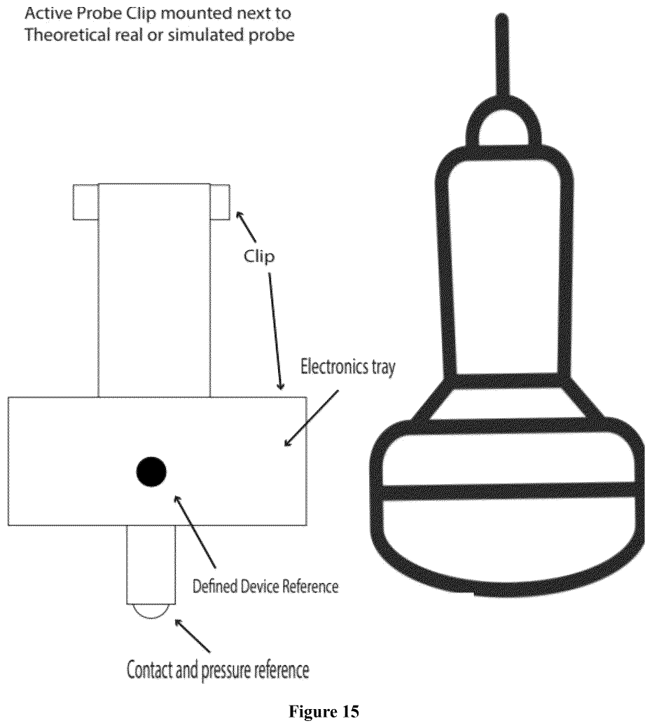

FIG. 15 shows a configuration of a tracking clip that would fit over a real or simulated probe. The same technology could be made directly into a simulated probe or device. In some devices, the use of contact information is not needed. FIG. 8 shows the relationship of the device with the clip and probe in relationship to the body with the AC electromagnetic internal reference. FIG. 13 shows the relationship of the operating system with control interface, display and networking, and additional devices that can be oriented to the internal reference system of the body and the limb structures.

The training system and method allows for the dynamic movement of the manikin and/or simulated body or standardized patient as they would be in real life while being scanned and treated. This allows for devices to be used together as they would be used in a real clinical environment rather than as separate training tasks. All training for ultrasound now relies on the body object to be static and not move while training. The Ultrasound training is currently limited on the number of devices it can coordinate in the simulation, while this system allows the use of devices in the training to equal the number that would be required in real clinical treatment of a patient providing greater training realism and complexity.

The practitioner should know the location and placement of the device on an approximation of human anatomy, the problem addressed in the patent is getting the imaging to align for that position since there is no internal anatomy to reference in the system that approximates human anatomy.

An application for a mobile device or software on computer or internet-based interface will do the mathematical calculations to:
1. Determine device orientation and position relative to the body.
2. Orient and composite the image in real time or other presentation for simulated devices.

Using an algorithm, AI or other methods to integrate the alignment and take the reference information to determine the location in three- or two-dimensional space in relationship to the manikin to the normal use within the action of the procedure with the device.

The device(s) and displays can be connected by wire or wirelessly.

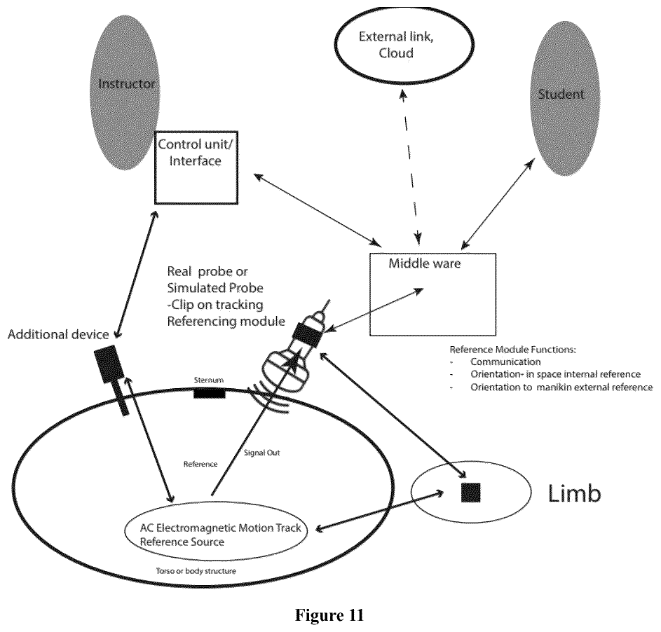

Hardware clip or embedded includes the use of battery or direct connect power supply to power the AC Electromagnetic reference system, internal to the body structure and or limb with related micro controller or computing device system. The internal reference will interact with the defined device reference outlined in FIG. 11. The tracking clip which is either clipped onto the medical device or embedded in it contains a power source or cord to power, a micro controller that can manage the electronic device such as gyro/time of flight or other electronics to track the roll, pitch and yaw relative to the defined device reference, a contact, pressure point (which may be optional on some medical device applications) and communications either tethered or wireless. In combination with the pressure sensing layer, the AC Electromagnetic reference system allows for accuracy to under 1 mm.

The data generated is used to orient visual data in orientation consistent with the ultrasound device within a device with a screen or projection that allows the student to see it in real time and the instructor to control it as outlined in FIG. 13. Other devices may just track position, placement parameters, or trigger events such as playing audio for auscultations based on the data provided.

One device or multiple devices can be used in any combination to better reflect the use of complementing technology found in real world clinical applications. The system allows data to be collected on the use of one or more devices used at a given time to present information and feedback as directed by the instructor to the student, and for instructor to measure the performance of the student.

Use of haptics with the devices can be embedded in the body or limb structure to complement the feedback to the student. Haptics can be triggered based on the proximity of the foreign body to key areas during the training. Such haptics include but are not limited to the discharge of fluids or gases, vibrations, pulses, and audio sounds, that would represent expected bodily response in relationship with the use of the device on the body for a given clinical procedure. This haptics are managed automatically or by the instructor to respond based on the proximity or activity of the object to a key area.

### Evaluation of the Compressions

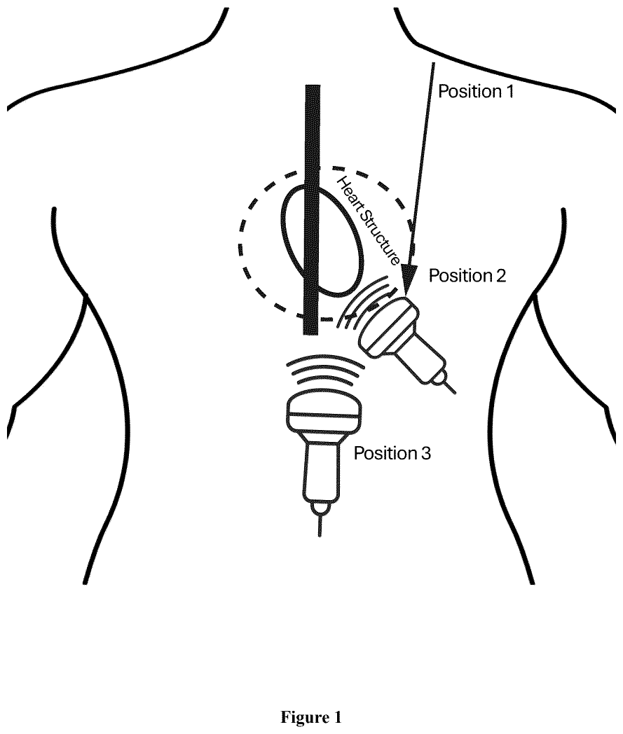

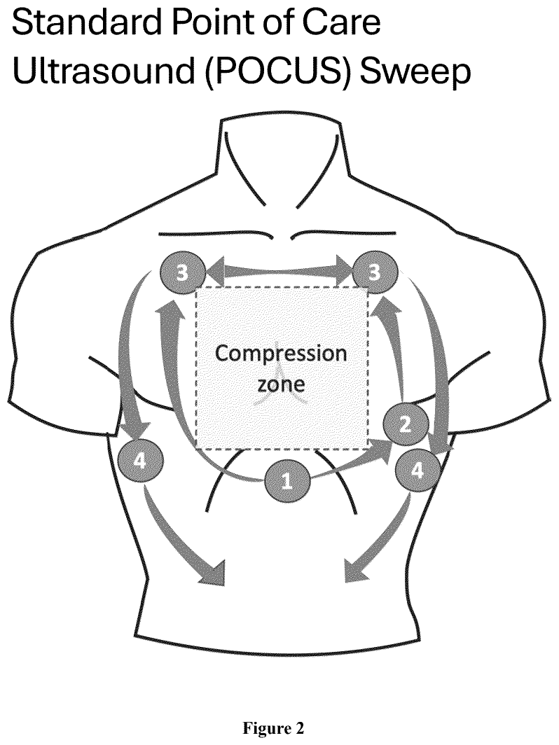

As shown in FIG. 1, the probe, real or simulated, moved on the chest in a manner to locate the heart. In FIG. 1 the probe moves from position 1 to 2 to intercept the heart. In the POCUS protocol, as shown in FIG. 2, the probe must be able to move around the chest as needed for an extensive evaluation. The system will be able to accommodate both rapid and longer scan methods as outlined.

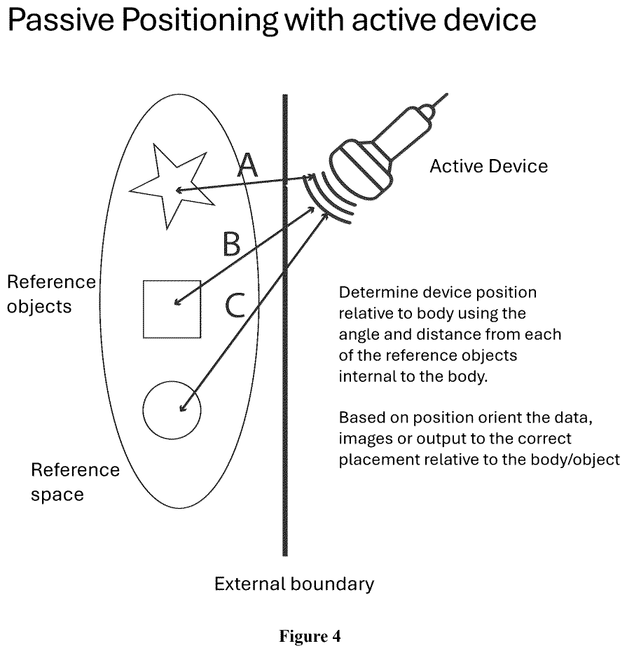

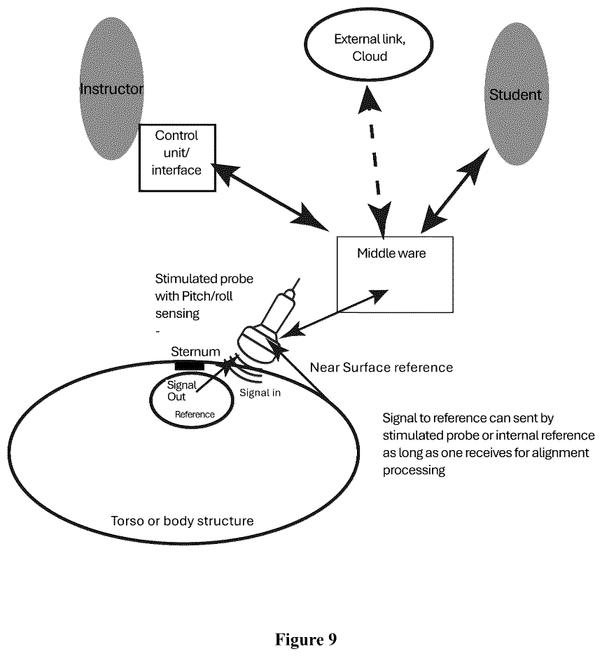

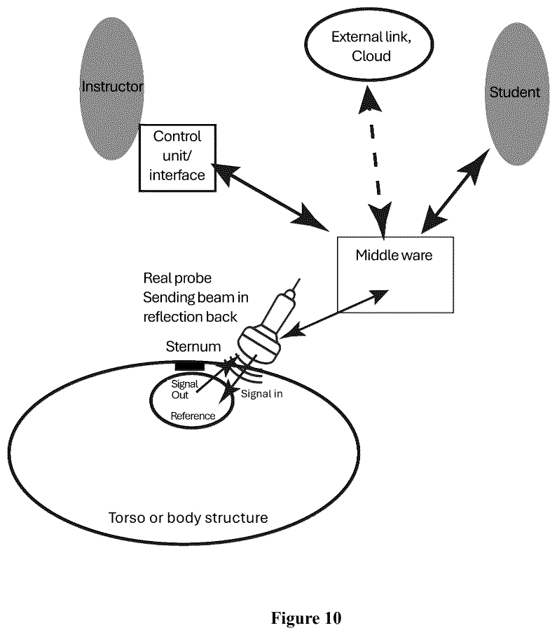

Once the right location and angle of the probe are achieved FIG. 4 in orientation of the patient and anatomy selected by the instructor, the effectiveness of compressions can be evaluated through the imagery presented though the middleware FIG. 9 and FIG. 10 to provide the clinical representation needed to support the training evolution. In the case of a real ultrasound probe the beam will use the internal structure image interpreted through the middleware to determine the placement and orientation and then determine how to present the correct image for the case in that position and orientation. In the case of the simulated probe, the internal beacon(s) and/or other surface and/or external references will link or be sensed by the simulated probe to use internal gyroscopic, time of flight or other related technology to pass onto the middleware to calculate correct position and orientation of the probe and then determine how to present the correct image for the case in that position and orientation. The system has the ability to move with the patient in the room, in a clinical environment or out in the field during training in real time and be part of a full body, partial body or skills trainer representation.

The system also has the capacity to show devices inserted into the body that can include such things as chest needles for chest decompression, chest drains, and trachea placement. It can also be used for non-torso presentations related to stroke to evaluate carotid front and rear, for example, blood entrapment related to compartment syndrome in limbs from crushing events as part of extraction training.

Images, virtualized/generated or taken from real cases, still and moving, including doppler and other image representations related or generated with the use of ultrasound used to represent normal and abnormal states such as impact on ventricles from compression, fluids, blood leakage, third spacing or related impacts, movement and non-movement of lung such sliding and related actions at boundary areas, patterns that are consistent with infection in the lung tissues, fluids in the lung tissue and pneumothorax, heart function like fibrillation, fine fibrillation and other heart function problems seen by ultrasound which may or may not synchronize with ECG wave forms or lack of wave forms. Movement like valve movement internal to the heart, including defects, blood clots and leakage, ejection with doppler and other blood flow information seen on ultrasound. These images represented will be selected by the instructor to reflect the conditions of the case of the simulation, presented relative the position and orientation of the probe real or simulated and reflect changes or impacts related to the action of the student or practicing participant in the form of changes in the imaging and or other feedback used during the training such as ECG traces or pulse for example.

### Pulseless Electrical Activity (PEA)/Asystole

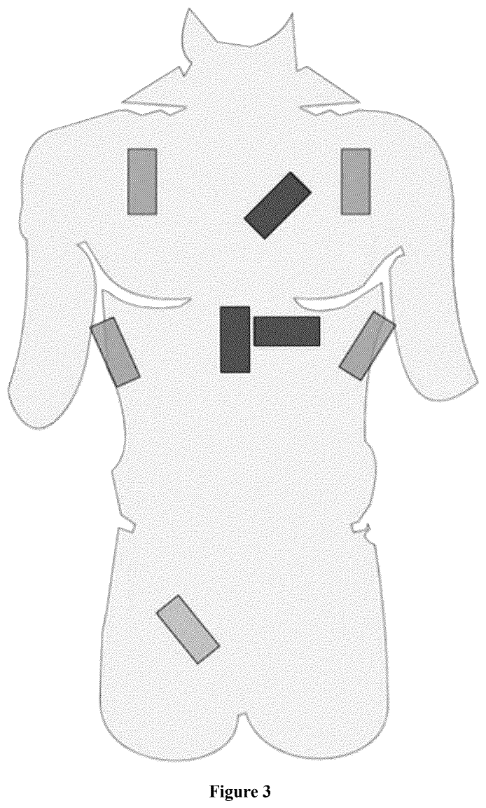

A full POCUS scan with the ultrasound device making sweeps around the compression site is shown in FIG. 2. This allows for a fully detailed evaluation of the patient as outlined in Blankco et al. The algorithm provided or related in purpose to that in FIG. 3 represents one strategy for the workflow. A simplified method of evaluation may also include placing the ultrasound about 2-3 centimeters below the xyphoid process and angling up to the heart to check the status of the heart in a more limited evaluation as outlined in the Easy Fast strategy for sepsis detection. In both strategies, the training equipment needs to track and present the information that support realistic movement around the patient during the cardio-respiratory event and be able to present the images and data that would be expected within a location and orientation that practitioner or student would make accurate interpretations to take actions that would benefit the patient and improve the likelihood of survival.

POCUS best practices are summarized in FIG. 3. POCUS should only be used during rhythm check and should not interfere with CPR efforts. The sonographer should prepare the curvilinear or the phased array probe so that image acquisition lasts 10 seconds. When the sonographer clicks the ‘acquire’ button, it usually records only 3 seconds of the scan and may not be enough time for the sonographer to record. The sonographer should appoint a ‘Time Keeper’ so that POCUS lasts less than 10 seconds. On pulse check ONLY acquire. The sonographer should interpret images when chest compressions have resumed. During chest compressions, the sonographer can look at extra cardiac images such as Lung/Abdomen/Vascular. The sonographer should communicate results with the rest of the team and should continue to obtain more scans once the sonographer has returned spontaneous circulation to verify the sonographer's impression or to change management.

Other information such as points of compression relative to the heart, for example hand chest compressions, can be sensed in the structure and include location, but also depth rate and release, and other related quality factors such as ventilations and ECG signals, whether real electronic signals or simulated. The body may be physical in nature but complemented, enhanced or augmented with augmented or virtual reality. Placement of electrodes for ECG, defibrillation pads and AED pads as well automated chest compression devices, pulse oximetry, capnography, blood pressure, glucometer and temperature can be part of the inclusive device sensing and or data presented in the training feedback during the training. This information can support both pre-briefing and debriefing in part or integrated into a unified management system linking with middleware directly or with various wireless methodologies to be used or stored locally or outside of the training on the internet or cloud outside of the local site. The devices in FIGS. 1, 2, 5, 9, and 10 can use one or more methods to link and sense, from acoustics, electronics including but not limited to direct wires, Bluetooth, Wi-Fi, LoRA radio or by light such as but not limited to infrared.

The devices function in a manner to allow POCUS and other related procedures mentioned above to be conducted while the probes are moved, orient to the presenting manikin full or partial body, or standardized patients with or without a compressible chest. In the case of non-compressible chests, force sensing can be employed.

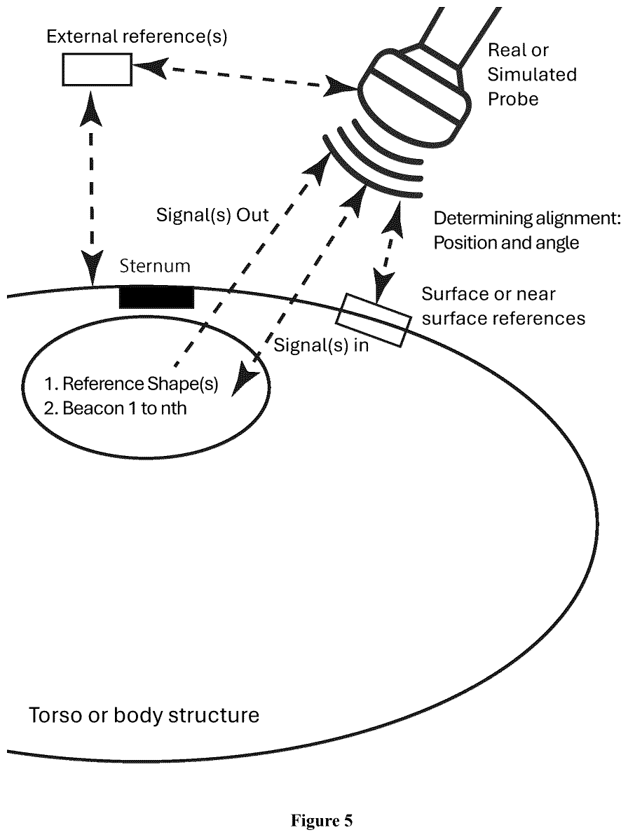

In FIG. 5 the operative field can use a mix of sensing and feedback to determine hand location, such as but not limited to capacitance sensing, pressure devices, time of flight devices and similar devices depending on the manikin structure or cover used in the case of standardized patients.

The manikin full body or in part and covering structures are a combination of urethanes, silicones, vinyl and thermal plastic TPE to vary to meet the needs of the body structure type to be employed to support the training system.

The system and method can support use in the classroom, simulation lab or clinical, varied clinical environments such as but not limited to hospitals, ambulances/transports, nursing homes, filed medical units, prolonged field care, and field environments like homes, outdoor sites local or remote, simulated battlefield environments.

The system and method's pressure sensing layer addresses a common problem in medical simulation. Namely, defining the location of a medical device or a hand on a simulated human form, a simulated human body part, a human form, or a human body part. The human form or body part may belong to a standardized patient. Defining location in these contexts is particularly important to ensure proper alignment of anatomical structures. The system and method may provide feedback regarding proper alignment to anatomical structures, the feedback comprising, haptic feedback, acoustic feedback, virtual or augmented reality feedback, or artificial intelligence-based feedback.

When pressure is applied to the pressure sensing layer, the system and method can determine the force vector direction of the pressure. Accordingly, the direction and angle for the relative or absolute pressure, as well as the orientation of the contacting object, can be determined. The orientation of the contacting object can be determined with six degrees of movement, i.e., six degrees of freedom.

Secondary devices like needles or other intrusive equipment can be used in combination with virtualized ultrasound. It can be referenced in one or more ways such as the secondary device using the pressure sensing layer and internal device reference for gross procedures with lower resolution or in combination with the pressure sensing layer an AC magnetic field reference system that will allow for accuracy to under 1 mm. One example of such systems includes but is not limited to units manufactured currently manufactured by Polhemus.

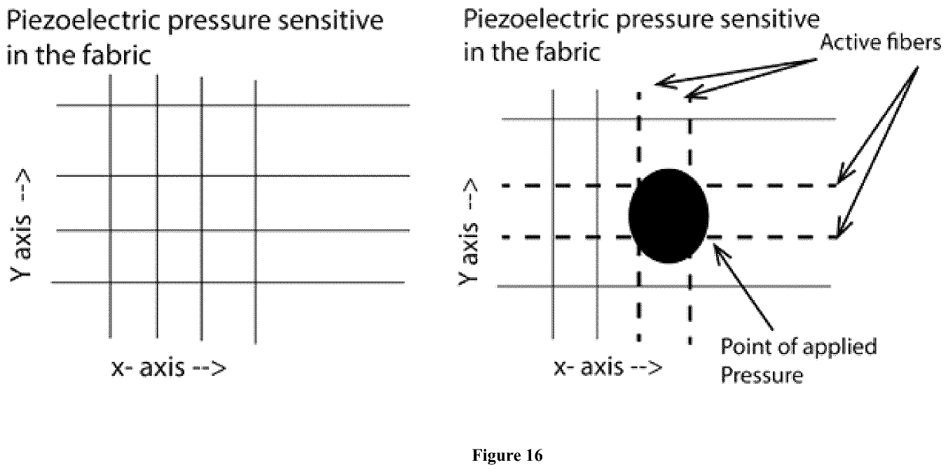

The pressure sensing layer can define a grid and determine the amount, location, and vector of pressure being applied to a point being 1 centimeter square or less, as shown in FIG. 16.

Palpation, the use of the hands of the practitioner to probe tissue and/or organs in the general area or palpate organs and glands to determine the feel and consistency. Evaluation examples, abdominal exams, localized glands. The pressure sensing layer will be used in substructures under the surface to develop heatmaps on organs and glands to show practitioners the impact and effectiveness of their efforts to provide visual feedback to support the development and improvement muscle memory of practitioners. The surface heatmaps and the organ heat maps will be able to provide a detailed presentation that is not currently available. The substructures will be able to physically alter to change the feel and consistency to provide the ability to present realistic alternative abnormal medical conditions that exist in real patients.

Regarding hemorrhage control, working with the existing Training Bridge Wound Cell the hemorrhage control trainer that provides absolute pressure feedback, or other wound training systems. Simulations of Applied pressure used during the initial phase to stop or slow bleeding prior to packing the wound for clot formation. Force vector-Use pressure sensing layer to establish a heatmap and force vectors to help train practitioners on proper application of force projection to increase the efficiency of how they apply force. This provides real-time feedback to allow practitioners to change body mechanics and see the impact of the changes. For wound packing and cap dressing, the correct filling of wound with packing to support clot formation and cap dressing to hold packing. Pressure vector is needed to ensure not only consistent absolute pressure as measured in the wound cell, but the wound cavity is filled, and force vector is optimized for the development of endurance of the practitioner. For the cap dressing ensuring the force of the dressing is in line with the wound packing. A heatmap and force vector to provide real-time feedback on placement and effectiveness of the cap dressing.

Regarding tourniquets, initial application force applications vary by manufacturer, and practitioners need to train and understand the impact of the equipment they use. Force vector determination ensures the focal point of the force of the device is aligned with the major blood vessels for the specific anatomical points used. The heatmap shows the relative force application on the circumference of the limb profile. In the context of long-term management, vessel management and prolonged casualty care, the heatmap provides training on the proper securing and releasing of tourniquets and related devices is critical on salvaging limbs and avoiding onset of cardiac arrest from the rapid and uncontrolled release of electrolytes, known as compartment syndrome. The heatmap shows the relative force application on the circumference of the limb profile.

Objects such as body parts and devices may be identified using a combination of one or more of: 1) pressure points using of X-Y coordinates communicated by the pressure sensing layer; 2) heatmaps based on the foot print patterns from the face of the object pressing on the pressure sensing fabric which, can be assisted with local edge detection based artificial intelligence or other algorithms; 3) RFID tagging to determine the general geographic area in operation to separate multiple objects on the surface or body area in combination with 1 and/or 2 to provide a higher level of detail and device identification separation; and 4) an AC magnetic field referencing used in combination with 1, 2, and/or 3 with the pressure sensing layer to one or more devices higher accuracy of 1 mm or less.

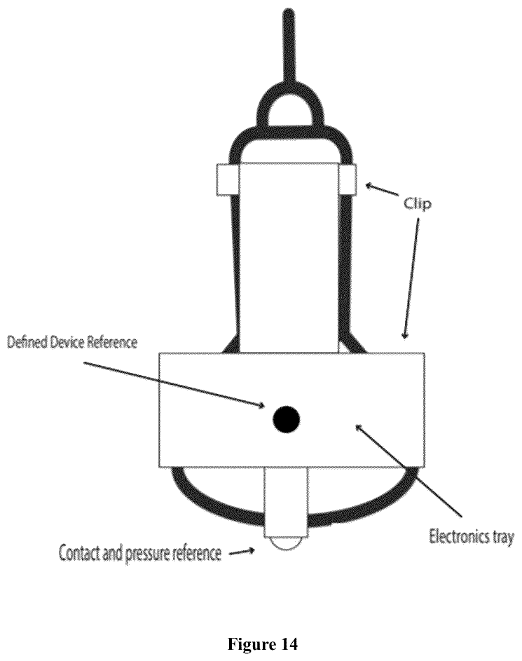

The device, whether simulated real, is located using existing telemetry or with the use of a clip-on device supporting these elements used separately or in combination with the data from the pressure sensing layer. FIG. 14 provides an outline of the general relationship to an ultrasound device. The device may be configured without such attachments as in the case of a single stethoscope simulated device. The system and method can capture six degrees or more of movement, i.e., six degrees of freedom, time of flight, absolute pressure on the face of the device, and AC magnetic field referencing, all with respect to the device.

The surface and related body will have a unit to collect the data from the pressure sensing layer after the initial local processing prior to sending it to the control unit either wirelessly or hardwired. Data from the device uses wireless or hardwire connection to relay the data collected or generated related to the movement and function of the device to a control unit. The control unit integrates the data from the pressure sensing layer and supports references with data from the device(s). Based on the device orientation, location and application to the related surface or body the feedback in the form of any combination, images, video, haptics or audio appropriate for a clinical device in the same place, orientation and application will be presented in a virtualized presentation consistent for the device with the associated haptics, such as a pulse or chest movement.

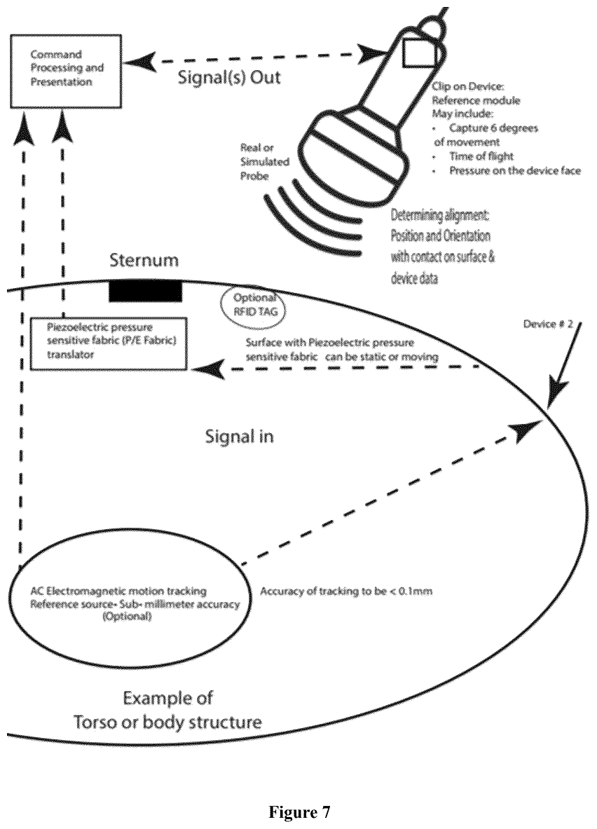

The control interface also allows for the scenario dynamics to change to present both normal and abnormal states that would expect or unexpected for the given simulated event. The coordination of the control system also supports the ability to isolate the specific function of the scenario or run it concurrently with other functions that may be part of the larger simulation to support providing a more realistic simulated clinical experience to the participant. FIG. 7 provides a relationship between the general operational parts and optional parts that may be used to enhance the simulation for a given clinical simulation.

This invention provides a medical system. In one embodiment, said system comprises a) a pressure sensitive layer for providing the location of an object in contact with a surface; and b) at least one pressure sensing surface or near-surface reference for differentiating multiple objects in contact with said surface.

In one embodiment, the amount, location, and vector of the pressure being applied to said pressure sensitive layer is displayed on a heatmap.

In a further embodiment, said heatmap is used to identify and differentiate an object of interest from incidental contact with other objects.

In one embodiment, said system comprises at least one external reference.

In a further embodiment, said at least one external reference is a tracking clip.

In a further embodiment, said tracking clip comprises a Radio Frequency Identification Detection (RFID) tag.

In a further embodiment, said RFID tag has a detection area greater than 1 cm².

In one embodiment, said system is used to simulate medical situations including cardiopulmonary resuscitation cardiac prolapse, cardiopulmonary resuscitation fine ventricular fibrillation, cardioversion shock, palpation, hemorrhage control, wound packing, cap dressing, tourniquets, vessel management, and prolonged casualty care.

This invention further provides a method of training individuals in medical techniques using the system of this invention. In one embodiment, the method is used to simulate medical situations including cardiopulmonary resuscitation cardiac prolapse, cardiopulmonary resuscitation fine ventricular fibrillation, cardioversion shock, palpation, hemorrhage control, wound packing, cap dressing, tourniquets, vessel management, and prolonged casualty care.

This invention further provides a training system. In one embodiment, said training system comprises at least one pressure sensing layer.

In one embodiment, said at least one pressure sensing layer comprises one or more layers of pressure sensitive fabric.

In one embodiment, said at least one pressure sensitive fabric is a piezoelectric pressure sensitive fabric.

In one embodiment, said at least one pressure sensing layer is encapsulated or integrated with silicone, urethane-based resins, and thermal plastic.

In one embodiment, said at least one pressure sensing layer is set in or cast in resins, silicones, and plastics, creating a “sandwich”.

In one embodiment, said sandwich comprises one or more layers of pressure sensitive fabric separated by one or more material.

In one embodiment, said at least one pressure sensing layer is integrated with, sleeved over, or placed on a simulation manikin or skills trainer.

In one embodiment, said at least one pressure sensing layer is sleeved onto the form or body parts of a human for low-risk procedures.

In one embodiment, said at least one pressure sensing layer is sleeved onto the form or body parts of a standardized patient for low-risk procedures.

In one embodiment, said at least one pressure sensing layer detects the application of pressure of a device, providing the location of the object applying the pressure.

In one embodiment, said object is a body part selected from a list comprising a hand or

torso.

In one embodiment, said object is a device selected from a list comprising an ultrasound probe, a stethoscope, or a similar device.

In one embodiment, said training system further comprises at least one external reference, and at least one surface or near-surface reference.

In one embodiment, said training system further comprises at least one internal reference.

In one embodiment, said training system further comprises at least one medical device, whose location may be defined by said training system.

In one embodiment, said at least one medical device may be an Ultrasound probe, stethoscope, or other medical device.

In one embodiment, said training system further comprises a feedback module that indicates whether a medical device or hand is in a desired location.

In one embodiment, said feedback module further provides feedback such as output on a graphical heatmap, haptic feedback, acoustic feedback, virtual or augmented reality feedback, or artificial intelligence-based feedback.

In one embodiment, said training system comprises a tracking clip to filter out medical devices when more than one medical device is being used. Filtering out certain medical devices from detection by the training system helps to differentiate the contact between the medical device of interest and the pressure sensing layer from incidental contact with other medical devices.

In one embodiment, said training system further provides information comprising images, video, and audio based on said medical device's location.

In one embodiment, said training system further provides updated information comprising images, video, and audio if the location of said medical device changes.

In one embodiment, said training system further provides updated information in response to input from a medical instructor. For instance, medical instruction may provide information simulating cardioversion shock if the trainee detects ventricular fibrillation.

In one embodiment, said training system further provides information comprising images, video, and audio based on the location of the practitioner's hands or other body parts.

In one embodiment, said training system provides updated information comprising images, video, and audio if the location of the practitioner's hands or other body parts changes.

In one embodiment, said training system automatically provides updated information using artificial intelligence.

In one embodiment, said training system provides updated information in response to input from a medical instructor.

In one embodiment, said tracking clip comprises a Radio Frequency Identification Detection (RFID) tag.

In one embodiment, said RFID tag has a detection area greater than 1 cm².

In one embodiment, said at least one pressure sensing layer comprises two or more layers of pressure sensitive fabric separated by one or more materials.

In one embodiment, said materials separating the layers of pressure sensitive fabric include silicones, urethane-based resins, and thermal plastic.

In one embodiment, said at least one pressure sensing layer is placed over a simulated human form or body part.

In one embodiment, said at least one pressure sensing layer is worn by a medical trainer.

In one embodiment, said at least one pressure sensing layer is worn by a standardized patient.

This invention further provides a training system. In one embodiment, said training system comprises a) a pressure sensing layer for providing the location of an object in contact with a surface; b) at least one pressure sensing surface or near-surface reference for differentiating multiple objects in contact with said surface; c) a non-transitory computer readable storage medium storing computer program instructions defined by modules of a computerized system; and d) at least one computing unit coupled to said non-transitory computer readable storage medium, at least one computing unit configured to execute computer program instructions defined by said modules of computerized system, wherein said modules comprising: i) a receiving module for receiving data from said pressure sensing layer; ii) a determination module for determining the pressure location and surface pressure pattern on said pressure sensing layer; iii) an orientation module for determining the orientation of said object relative to said surface; and iv) a display module for displaying said location and orientation of said object.

In one embodiment, said training system further comprises one or more additional pressure sensing layers for determining the direction and force vector of an applied force on said surface.

In one embodiment, said object comprises at least one internal reference for determining said orientation of said object relative to said surface.

In one embodiment, said training system further comprises at least one external reference.

In one embodiment, said object is selected from a body part or a medical device.

In one embodiment, said object is a stethoscope or a device having no relative internal orientation.

In one embodiment, said object is an ultrasound probe having a relative internal orientation.

In one embodiment, said at least one internal reference is an AC electromagnetic reference.

In one embodiment, said at least one internal reference of the training system provides the location and orientation of an object inserted beneath said pressure sensing layer.

In one embodiment, said training system is an ultrasound cardiorespiratory arrest training system.

In one embodiment, said training system is an ultrasound training system.

In one embodiment, said training system is an auscultation training system.

In one embodiment, the training system is a compression training system.

In one embodiment, said compressing training system is a hemorrhage control training system.

In one embodiment, said training system further comprises a feedback module that indicates whether said object is in a desired location and has a desired orientation relative to said surface.

In one embodiment, said feedback module indicates whether said object is in a desired location and has a desired orientation using at least one of video feedback, haptic feedback, acoustic feedback, virtual or augmented reality, or artificial intelligence.

In one embodiment, said pressure sensing layer comprises one or more layers of pressure sensitive fabric.

In one embodiment, said pressure sensitive fabric is a piezoelectric fabric.

In one embodiment, said one or more layers of pressure sensitive fabric is encapsulated or integrated with one or more materials including silicones, urethane-based resins, and thermal plastic.

In one embodiment, said pressure sensing layer comprises two or more layers of pressure sensitive fabric separated by one or more materials.

In one embodiment, said one or more fabric separating materials include silicones, urethane-based resins, and thermal plastic.

In one embodiment, said pressure sensing layer is worn by a subject selected from a simulation manikin, a standardized patient, or a medical instructor.

In one embodiment, said at least one external reference is a tracking clip for measuring the relative orientation of said object to said surface.

In another embodiment, said tracking clip reads a Radio Frequency Identification Detection (RFID) tag.

This invention further provides a method of training individuals in medical techniques. In one embodiment, said method comprises the steps of: a) receiving information from at least one pressure sensing layer; b) receiving information from at least one external reference; c) receiving information from at least one surface or near-surface reference; d) determining the location and surface pressure of an object on said pressure sensing layer; e) determining the location, orientation, and movement of said object; and f) displaying a visual or audio output based on said location, orientation, and movement of said object.

In one embodiment, said object in said method is an ultrasound probe.

In one embodiment, said method further comprises the step of receiving information from at least one internal reference.

In one embodiment, said internal reference is an AC electromagnetic reference for internal tracking of objects that have been inserted beneath said pressure sensing layer.

In one embodiment, said method further comprises the step of indicating whether said object is in a desired location using at least one of video feedback, haptic feedback, acoustic feedback, virtual or augmented reality, or artificial intelligence.

This invention further provides an ultrasound training system. In one embodiment, said ultrasound training system comprises: a) a pressure sensing layer; b) at least one external reference; c) at least one surface or near-surface reference; d) a non-transitory computer readable storage medium storing computer program instructions defined by modules of a computerized system; and e) at least one computing unit coupled to said non-transitory computer readable storage medium, said at least one computing unit configured to execute computer program instructions defined by said modules of computerized system, said modules further comprising: i) a receiving module for receiving data from said pressure sensing layer and references; ii) a determination module for determining the location and orientation of an ultrasound probe; iii) a display module for displaying said location and orientation of said ultrasound probe; and iv) a feedback module for indicating whether said ultrasound probe is in a desired location and has a desired orientation.

In one embodiment, said ultrasound training system further comprises one or more additional pressure sensing layers for determining the direction and force vector of an applied force on said surface.

In one embodiment, said ultrasound training system further comprises at least one internal reference, wherein said at least one internal reference is an AC electromagnetic reference.

This invention further provides an auscultation training system. In one embodiment, said auscultation training system comprises: a) a pressure sensing layer; b) at least one surface or near-surface reference; c) a non-transitory computer readable storage medium storing computer program instructions defined by modules of a computerized system; and d) at least one computing unit coupled to said non-transitory computer readable storage medium, said at least one computing unit configured to execute computer program instructions defined by said modules of computerized system, said modules further comprising: i) a receiving module for receiving data from said pressure sensing layer and references; ii) a determination module for determining the location and orientation of a stethoscope; iii) a display module for displaying said location and orientation of said stethoscope; and iv) a feedback module for indicating whether said stethoscope is in a desired location and has a desired orientation, wherein said stethoscope is used as an auscultation trainer using one or more stethoscopes, or in conjunction with an ultrasound trainer.

This invention further provides a training system. In one embodiment, said training system comprises: a) a first pressure sensing layer; b) a second pressure sensing layer for determining object position and relative force vector of tissue compression; c) a non-transitory computer readable storage medium storing computer program instructions defined by modules of a computerized system; and d) at least one computing unit coupled to said non-transitory computer readable storage medium, said at least one computing unit configured to execute computer program instructions defined by said modules of computerized system, said modules further comprising: i) a receiving module for receiving data from said pressure sensing layers; ii) a determination module for determining the location and orientation of pressure applied by an object; iii) a measuring module for determining an absolute measure of applied pressure beneath said first and second pressure sensing layers; iv) a display module for displaying said location and orientation of said applied pressure; and v) a feedback module for indicating whether said relative force vector is in the desired direction, whether an absolute pressure meets a minimum desired pressure, whether said absolute pressure is in a desired location, and whether said absolute pressure is in a desired orientation.

In one embodiment, said training system is a hemorrhage control training system.

In one embodiment, said training system is a palpation training system.

In one embodiment, said training system is an object placement and pressure training system.

In another embodiment, said object in said placement and pressure training system is an airway device or gynecological procedural device.

### CROSS-REFERENCE TO RELATED APPLICATIONS

This application claims the benefit of U.S. Ser. No. 63/665,278, filed Jun. 28, 2024, and U.S. Ser. No. 63/665,277, filed Jun. 28, 2024. The entire contents and disclosures of the preceding applications are incorporated by reference into this application. Throughout this application, various publications are cited. The disclosures of these publications in their entireties are hereby incorporated by reference into this application to more fully describe the state of the art to which this invention pertains.

1. A training system comprising:
a. a pressure sensing layer for providing the location of an object in contact with a surface;
b. at least one pressure sensing surface or near-surface reference for differentiating multiple objects in contact with said surface;
c. a non-transitory computer readable storage medium storing computer program instructions defined by modules of a computerized system; and
d. at least one computing unit coupled to said non-transitory computer readable storage medium, at least one computing unit configured to execute computer program instructions defined by said modules of computerized system, wherein said modules comprising:
i. a receiving module for receiving data from said pressure sensing layer;
ii. a determination module for determining the pressure location and surface pressure pattern on said pressure sensing layer;
iii. an orientation module for determining the orientation of said object relative to said surface; and
iv. a display module for displaying said location and orientation of said object.

2. The training system of claim 1, further comprising one or more additional pressure sensing layers for determining the direction and force vector of an applied force on said surface.

3. The training system of claim 1, wherein said object comprises at least one internal reference for determining said orientation of said object relative to said surface.

4. The training system of claim 1, wherein said object is selected from a body part or a medical device.

5. The training system of claim 1, wherein said training system is an ultrasound cardiorespiratory arrest training system.

6. The training system of claim 1, wherein said training system is a compression training system.

7. The training system of claim 1, further comprising a feedback module that indicates whether said object is in a desired location and has a desired orientation relative to said surface.

8. The training system of claim 1, wherein said pressure sensing layer comprises one or more layers of pressure sensitive fabric.

9. The training system of claim 8, wherein said pressure sensitive fabric is a piezoelectric fabric.

10. The training system of claim 8, wherein said one or more layers of pressure sensitive fabric is encapsulated or integrated with one or more materials including silicones, urethane-based resins, and thermal plastic.

11. The training system of claim 9, wherein said pressure sensing layer comprises two or more layers of pressure sensitive fabric separated by one or more materials.

12. The training system of claim 1, wherein said pressure sensing layer is worn by a subject selected from a simulation manikin, a standardized patient, or a medical instructor.

13. The training system of claim 1, wherein said at least one external reference is a tracking clip for measuring the relative orientation of said object to said surface.

14. A method of training individuals in medical techniques, comprising the steps of:
a. receiving information from at least one pressure sensing layer;
b. receiving information from at least one external reference;
c. receiving information from at least one surface or near-surface reference;
d. determining the location and surface pressure of an object on said pressure sensing layer;
e. determining the location, orientation, and movement of said object; and
f. displaying a visual or audio output based on said location, orientation, and movement of said object.

15. The method of claim 14, wherein said object is an ultrasound probe.

16. The method of claim 14, further comprising the step of indicating whether said object is in a desired location using at least one of video feedback, haptic feedback, acoustic feedback, virtual or augmented reality, or artificial intelligence.

17. An ultrasound training system, comprising:
a. a pressure sensing layer;
b. at least one external reference;
c. at least one surface or near-surface reference;
d. a non-transitory computer readable storage medium storing computer program instructions defined by modules of a computerized system; and
e. at least one computing unit coupled to said non-transitory computer readable storage medium, said at least one computing unit configured to execute computer program instructions defined by said modules of computerized system, said modules comprising:
i. a receiving module for receiving data from said pressure sensing layer and references;
ii. a determination module for determining the location and orientation of an ultrasound probe;
iii. a display module for displaying said location and orientation of said ultrasound probe; and
iv. a feedback module for indicating whether said ultrasound probe is in a desired location and has a desired orientation.

18. The ultrasound training system of claim 17, further comprising one or more additional pressure sensing layers for determining the direction and force vector of an applied force on said surface.

19. A training system, comprising:
a. a first pressure sensing layer;
b. a second pressure sensing layer for determining object position and relative force vector of tissue compression;
c. a non-transitory computer readable storage medium storing computer program instructions defined by modules of a computerized system; and
d. at least one computing unit coupled to said non-transitory computer readable storage medium, said at least one computing unit configured to execute computer program instructions defined by said modules of computerized system, said modules comprising:
i. a receiving module for receiving data from said pressure sensing layers;
ii. a determination module for determining the location and orientation of pressure applied by an object;
iii. a measuring module for determining an absolute measure of applied pressure beneath said first and second pressure sensing layers;
iv. a display module for displaying said location and orientation of said applied pressure; and
v. a feedback module for indicating whether said relative force vector is in the desired direction, whether an absolute pressure meets a minimum desired pressure, whether said absolute pressure is in a desired location, and whether said absolute pressure is in a desired orientation.

20. The training system of claim 19, wherein said system is a hemorrhage control training system, a palpation training system, or an object placement and pressure training system.
